(19) United States
(12) Patent Application Publication
(10) Pub. No.: US 2025/0213844 A1
(43) Pub. Date: Jul. 3, 2025

(54) SYSTEM AND METHOD FOR TAPERED LUER CONNECTOR FOR MEDICAL TUBING

(71) Applicant: KORU Medical Systems, Inc., Mahwah, NJ (US)

(72) Inventors: BRIAN CHRISTOPHER CASE, Lake Villa, IL (US); PAUL TARSIA, Ringwood, NJ (US); WESLEY HOWELL, Ringwood, NJ (US)

(73) Assignee: KORU Medical Systems, Inc., Mahwah, NJ (US)

(21) Appl. No.: 18/991,213

(22) Filed: Dec. 20, 2024

Related U.S. Application Data
(60) Provisional application No. 63/616,368, filed on Dec. 29, 2023.

(51) Int. Cl. A61M 39/10; A61M 5/142

(52) U.S. Cl. CPC A61M 39/10; A61M 5/142

(57) ABSTRACT

Provided is a tapered luer connector for medical tubing. The tapered lure connector has a tube head adapter formed of a resilient material providing: an inlet and opposite thereto an outlet with a longitudinal axis there between; a luer lock fitting formed proximate to the inlet and structured and arranged to engage a syringe; and a tapered member disposed at least proximately to the outlet, at least a portion of the tapered member angled down towards the outlet and providing an external engaging surface about a portion of the tube head adapter proximate to the outlet, the outer engaging surface obtusely angled away from the outlet with respect to the longitudinal axis. An associated method of use is also provided.

### CROSS REFERENCE TO RELATED APPLICATIONS

This application claims the benefit under 35 U.S.C. § 119(e) of U.S. Provisional Application No. 63/616,368 filed Dec. 29, 2023, and entitled SYSTEM AND METHOD FOR TAPERED LUER CONNECTOR FOR MEDICAL TUBING, the disclosure of which is incorporated herein by reference.

### FIELD OF THE INVENTION

The present invention relates generally to medical devices, and more specifically to medical tubing and systems and methods of maintaining sterility and eliminating the opportunity for random inter connection between tubing and a syringe by ensuring proper connections to syringes or other pharmaceutical reservoirs and pumps for the delivery of pharmaceutical products. In addition, the present invention minimizes the opportunity for use of third party or unintended medical tubing by employing a specific coupling system.

### BACKGROUND

Liquid pharmaceuticals are commonly delivered to patients through injection or infusion. Such delivery is generally made in one of two ways. The first is an immediate delivery from a health care provider in the form of a simple injection performed with a syringe and a needle directly disposed to the tissue of the patient. The second is gradual delivery, wherein a syringe or other reservoir is connected to specific medical tubing for delivery over time.

Frequently, syringe pumping systems such as Sealfon U.S. Pat. No. 5,741,227 (hereinafter “Sealfon '227”) teaching a Method of Sterile Preparation of IV Pump Syringe are used. Sealfon '227 teaches a number of advantages for maintaining the sterility of the tubing set 14, coupling the tubing set 14 to the syringe 12, and disposing the coupled syringe 12 within a syringe pump 10.

More specifically, Sealfon '227 teaches a disc 48 that is added to the tubing set 14 at the connector end interposed between the tubing set per se inlet 34 and a tubing engaging means 50. This disc 48 has been shown to provide and perform many advantages. For example, this disc 48 serves to elevate the tubing end from surfaces, thereby promoting the sterile condition of the tubing end, if and/or when it is set down when unconnected to a syringe.

In addition, the disc 48 provides a convenient brace for an operator's fingers during the coupling of the tubing set to a syringe—the disc 48 further preventing slippage of the fingers over the inlet 34 that again promotes sterility of the inlet 34. And further still, the disc provides a brace for the syringe pump 10, ensuring that when the coupled syringe 12 and tubing set 14 are disposed within the syringe pump 10, the disc 48 provides a brace against which the force of the syringe pump 10 serves to temporarily lock the coupled syringe 12 and tubing set 14 in place.

By using calibrated tubing and needles together with a constant pressure pump, such as the Sealfon '227 syringe pump, it is possible to virtually guarantee safe delivery of a liquid to tissue. When saturation of the tissue occurs the resistance to the flow increases and the system immediately self-adjusts by lowering the flow. Indeed, when the tissues have received enough liquid such that the pressure within the tissues equals the pressure from the pump, the flow of the administered liquid will cease. In terms of patient care, this is highly advantageous because it vastly reduces the chance for accidental over medication.

As with constant pressure systems, with constant flow systems calibrated tubing and needles are once again commonly used. However, with the constant flow system, as pressure increases at the receiving site the system will increase pressure to maintain the desired flow and over medication can result. Moreover, the focus on maintaining constant pressure can result in detrimental treatment for the patient.

However, a factor in both constant flow and constant pressure is use of the correct tubing. The availability of third-party tubing having a disc as taught by Sealfon '227 has introduced the possibility that despite best intentions, unwarranted tubing may be used that does not conform to the desired calibrations. As such unintended dosage may result and adversely affect the patient.

In addition, different tubing lines intended for different liquids providing different pharmaceuticals may be crossed leading to wrong substances being provided to the wrong tissues. As the use of the disc as taught by Sealfon '227 is highly useful in protecting the inlet and as a brace during tubing set connection, the disc itself does not aid in avoiding misconnections.

Moreover, as the use of the disc is not in and of itself protected, although competition within the marketplace is generally good, the availability of different tubing options utilizing a disc has introduced potential problems that may adversely affect patient health and care.

Hence there is a need for a tubing set alone, and tubing set with a syringe pump which overcome one or more of the above identified challenges.

### SUMMARY OF THE INVENTION

Our invention solves the problems of the prior art by providing novel systems and methods for a tapered luer connector for medical tubing.

In particular, and by way of example only, according to one embodiment of the present invention, provided is a tapered luer connector for medical tubing comprising: a tube head adapter formed of a resilient material providing: an inlet and opposite thereto an outlet with a longitudinal axis there between; a luer lock fitting formed proximate to the inlet and structured and arranged to engage a syringe; and a tapered member disposed at least proximately to the outlet, at least a portion of the tapered member angled down towards the outlet and providing an external engaging surface about a portion of the tube head adapter proximate to the outlet, the outer engaging surface obtusely angled away from the outlet with respect to the longitudinal axis.

In yet another embodiment, provided is a tapered luer connector for medical tubing comprising: a tube head adapter formed of a resilient material providing: an inlet and opposite thereto an outlet with a longitudinal axis there between; a disc element generally normal to the longitudinal axis and disposed between the inlet and the outlet; a luer lock fitting formed proximate to the inlet and structured and arranged to engage a syringe; and a tapered member disposed between the disc element and the outlet, the tapered member providing an external engaging surface about a portion of the tube head adapter proximate to the outlet, the outer engaging surface obtusely angled away from the outlet with respect to the longitudinal axis.

For yet another embodiment, provided is a tapered luer connector for medical tubing comprising: a tube head adapter formed of a resilient material providing an inlet and opposite thereto an outlet with a longitudinal axis there between and at least two members, a first member as a luer lock fitting adjacent to the inlet and structured and arranged to engage a syringe; and a second member as a circumferential tapered member adjacent to the outlet, the circumferential tapered member having an external engaging surface proximate to the outlet and sloping away from the luer lock fitting, at least a portion of the external engaging surface structured and arranged to engage a correspondingly angled receiver section of a base, the second member having a circumferential disc element extending normally with respect to the longitudinal axis.

Further still, in yet another embodiment provided is a base comprising: a body of resilient material with a longitudinal groove therein structured and arranged to receive at least a portion of the tapered luer connector, the longitudinal groove having: a longitudinal centerline; a first end and opposite thereto a second end; an inner receiver section tapering towards the longitudinal centerline and the second end, the inner receiver section angled to receive at least a portion of an outer engaging surface provided by a tapered luer connector; and an outer edge deflector section sloping across the longitudinal groove between the first end and the second end.

For still yet another embodiment, provided is a syringe pump for use with a tapered luer connector for medical tubing comprising: a housing structured and arranged to receive a syringe having a plunger movable along a barrel and an engaged tubing, the engaged tubing providing a head adapter providing an inlet and opposite thereto an outlet with a longitudinal axis there between with a luer lock fitting proximate to the inlet engaged to the syringe and a circumferential tapered member adjacent to the outlet, the circumferential tapered member having an external engaging surface proximate to the outlet and sloping away from the luer lock fitting, the engaging surface structured and arranged to engage a correspondingly angled receiver section of a base; a base having: a body of resilient material with a longitudinal groove therein structured and arranged to receive at least a portion of the tapered luer connector, the longitudinal groove having: a longitudinal centerline; a first end and opposite thereto a second end; an inner receiver section tapering towards the longitudinal centerline and the second end, the inner receiver section angled to receive at least a portion of an outer engaging surface provided by a tapered luer connector; an outer edge deflector section sloping across the longitudinal groove between the first end and the second end; and an actuator structured and arranged to engage the plunger of the syringe and drive the plunger towards the tubing, the force of the actuator further engaging the external engaging surface of the tapered member to the inner receiver section of the base.

And for yet another embodiment, provided is a method for using tapered luer medical tubing, comprising: providing a tube head adapter formed of a resilient material providing: an inlet and opposite thereto an outlet with a longitudinal axis there between; a luer lock fitting formed proximate to the inlet and structured and arranged to engage a syringe; and a tapered member disposed at least proximately to the outlet, at least a portion of the tapered member angled down towards the outlet and providing an external engaging surface about a portion of the tube head adapter proximate to the outlet, the outer engaging surface obtusely angled away from the outlet with respect to the longitudinal axis, the engaging surface structured and arranged to engage a correspondingly angled receiver section of a base; engaging the luer lock fitting of the tubing to a syringe having a barrel with a plunger disposed within and movable along the barrel, the angled engaging surface disposed away from the syringe; and disposing the syringe with engaged tubing within a pump having an actuator and a base, the base having: a body of resilient material with a longitudinal groove therein structured and arranged to receive at least a portion of the tapered luer connector, the longitudinal groove having: a longitudinal centerline; a first end and opposite thereto a second end; an inner receiver section tapering towards the longitudinal centerline and the second end, the inner receiver section angled to receive at least a portion of an outer engaging surface provided by a tapered luer connector; an outer edge deflector section sloping across the longitudinal groove between the first end and the second end; and an actuator structured and arranged to engage the plunger of the syringe and drive the plunger towards the tubing, the force of the actuator further engaging the external engaging surface of the tapered member to the inner receiver section of the base.

### BRIEF DESCRIPTION OF THE DRAWINGS

FIG. 1A is a front perspective view of a tapered luer connector in accordance with at least one embodiment of the preset invention;

FIG. 1B is a rear perspective view of a tapered luer connector in accordance with at least one embodiment of the preset invention;

FIG. 1C is a plain side view of a tapered luer connector in accordance with at least one embodiment of the preset invention;

FIG. 1D is a plain front view of a tapered luer connector in accordance with at least one embodiment of the preset invention;

FIG. 1E is a plain rear view of a tapered luer connector in accordance with at least one embodiment of the preset invention;

FIG. 2A is a front perspective view of a base for a tapered luer connector in accordance with at least one embodiment of the preset invention;

FIG. 2B is a rear perspective view of a base for a tapered luer connector in accordance with at least one embodiment of the preset invention;

FIG. 3A is a front perspective view of a tapered luer connector engaged to a base in accordance with at least one embodiment of the preset invention;

FIG. 3B is a rear perspective view of a tapered luer connector engaged to a base in accordance with at least one embodiment of the preset invention;

FIG. 3C is a top plain view of a tapered luer connector engaged to a base in accordance with at least one embodiment of the preset invention;

FIG. 3D is a side plain view of a tapered luer connector engaged to a base in accordance with at least one embodiment of the preset invention;

FIG. 3E is a side cut-through view of a tapered luer connector engaged to a base in accordance with at least one embodiment of the preset invention;

FIGS. 4A-4D are side plain views of a legacy luer being ejected by a base in accordance with at least one embodiment of the preset invention;

FIG. 5 is a flow diagram of a method of using a tapered luer connector in accordance with at least one embodiment of the preset invention; and

FIG. 6 is a presents a conceptual illustration of an infusion procedure utilizing a tapered luer connector in accordance with at least one embodiment of the present invention.

### DETAILED DESCRIPTION

Before proceeding with the detailed description, it is to be appreciated that the present teaching is by way of example only, not by limitation. The concepts herein are not limited to use or application with a specific system or method for a tapered luer connector for medical tubing, a kit with tapered luer medical tubing, or syringe pump for use with a tapered luer connector for medical tubing. Thus, although the instrumentalities described herein are for the convenience of explanation shown and described with respect to exemplary embodiments, it will be understood and appreciated that the principles herein may be applied equally in other types of systems and methods involving tubing with a tapered luer.

Within the medical industry small-bore connectors are commonly used to interconnect devices, tubing and accessories that deliver fluids and gases to patients. The small connectors typically provided on tubing and which are grasped by a person for connection and disconnection are generally referred to as luer fittings, or simply luers.

The actual connection element is more properly referred to as a luer taper, of which there are two types—locking and slipping. With a luer lock the tubing and medical or laboratory equipment are twisted together as at least one luer lock fitting has threads which bind one fitting to the other. With a luer slip, the fittings are just press fit together and held by friction. Where pressure is a factor, such as with syringes, pumps or the like, the luer lock is generally favored as the threading engagement provides a tighter leak free bond.

Despite the specific origin with reference to the connection, absent a specified type of connector, when reference is made to a “luer” it is commonly understood and accepted that it is the entire end connector that is being identified. As such, as referred to herein, a tapered luer is understood and appreciated to apply to the entire end fitting.

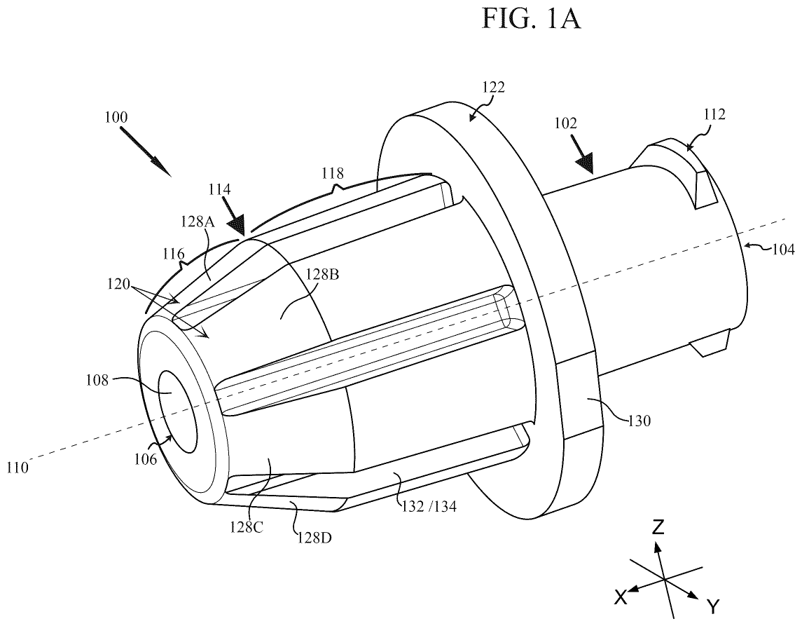

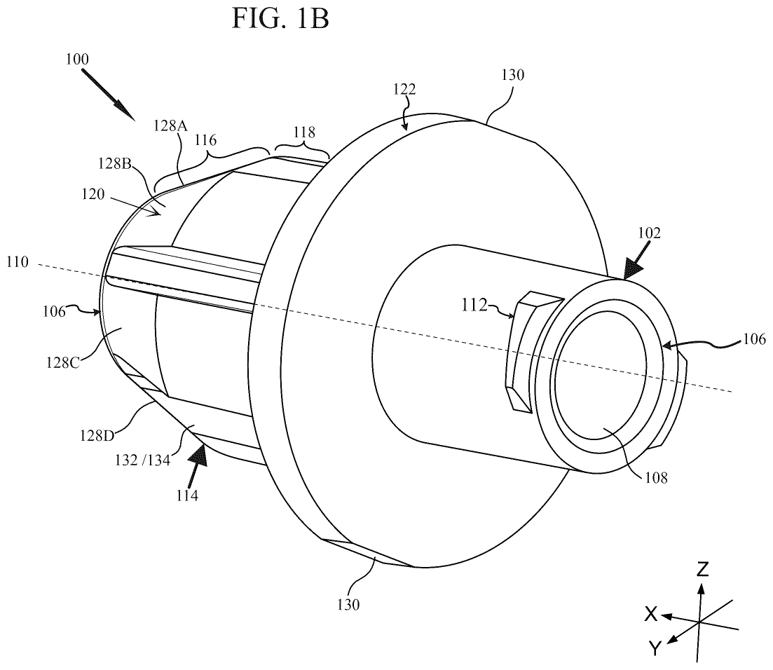

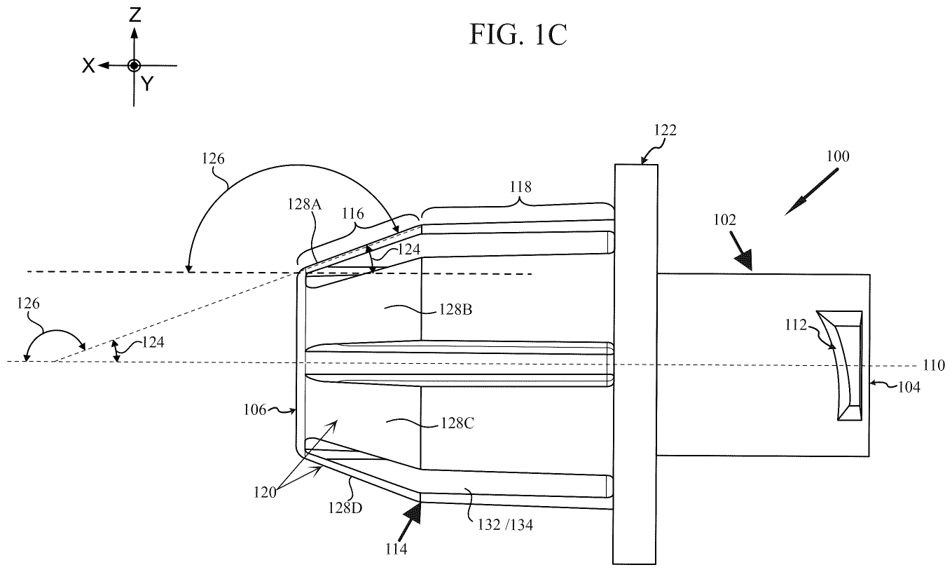

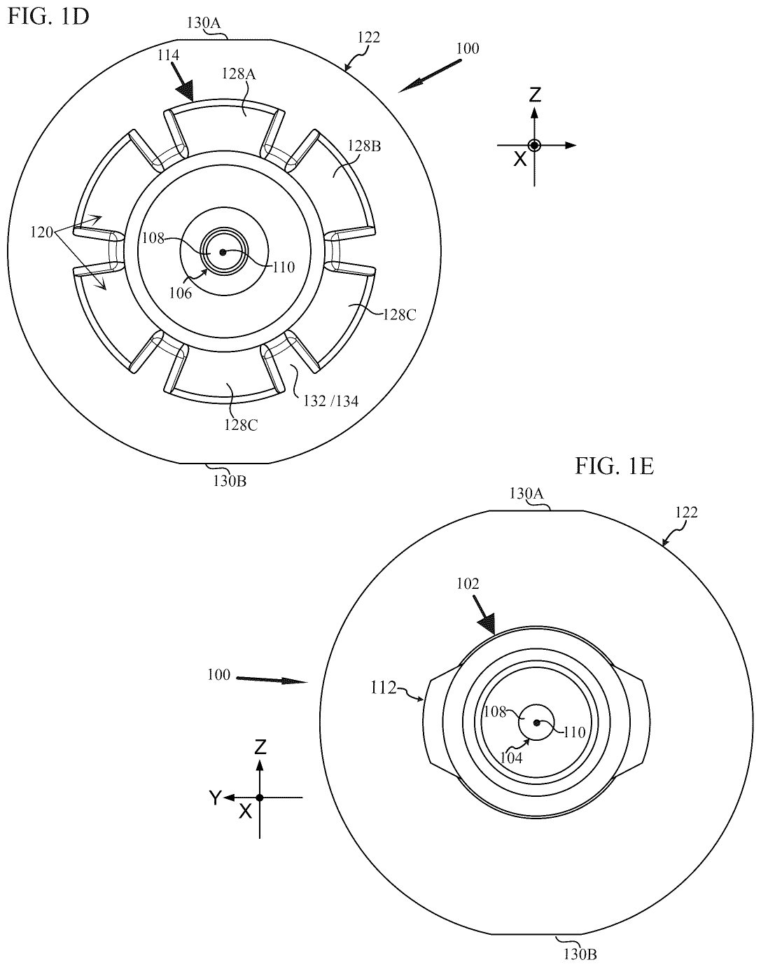

Turning now to the Figures, and more specifically FIGS. 1A, 1B, 1C, 1D and 1E, there is shown a tapered luer connector 100, hereinafter TLC 100, in accordance with at least one embodiment of the present invention. FIG. 1A is appreciated to be a front perspective. FIG. 1B a rear perspective, FIG. 1C a plain side view, FIG. 1D a front plain view and FIG. 1E a rear plain view. More specifically, for at least one embodiment, the TLC 100 is provided by a tube head adapter 102 formed from a resilient material, such as, but not limited to polycarbonate, plastic, ceramic, or other suitable material.

To facilitate the description of systems and methods for embodiments of TLC 100, the orientation of TLC 100 as presented in the figures is referenced to the coordinate system with three axes orthogonal to one another as shown in FIG. 1A. The axes intersect mutually at the origin of the coordinate system, which is chosen to be the center of TLC 100, however the axes shown in all figures are offset from their actual locations for clarity and ease of illustration.

As is shown, initially in the front and rear perspective views of FIGS. 1A and 1B, the tube head adapter 102 has an inlet 104 and opposite thereto an outlet 106 with a fluid passageway 108 about a first longitudinal axis 110 there between. A luer lock fitting 112 is provided proximate to the inlet 104. The outlet 106 of the tube head adapter 102 is structured and arranged to join with medical tubing-such as by gluing, bonding, welding, or other selected means or method of attachment. For the Figures as shown, the medical tubing has been omitted from being shown extending from the outlet 106 for ease of illustration and discussion, and appreciation of the TLC 100 itself as a distinct element. For at least one embodiment, a filter element (not shown) may be disposed within the fluid passageway 108 as set forth in U.S. Pat. No. 11,556,100 entitled CONNECTOR WITH FILTER, incorporated herein by reference.

Disposed proximate to the outlet 106 is a tapered member 114—rising away from the outlet 106 and generally towards the inlet 104. As shown, a first portion 116 of the tapered member 114 is angled up and away from the outlet 106 and rises to a second portion 118. For at least one embodiment, this second portion 118 is generally parallel to the first longitudinal axis 110. For at least one further embodiment, the second portion 118 may also be angled relative to the first longitudinal axis 110, and still further, the angle of the second portion 118 may be different from the angle of the first portion 116.

For at least one embodiment there is a clear visual distinction between the first portion 116 and the second portion 118, and yet for another embodiment the transition between the first portion 116 and the second portion 118 is gradual and not visually stark. Indeed, for at least one embodiment the TLC 100 may be described as having a “bullet” shape, such that it may be referred to as a “bullet luer”.

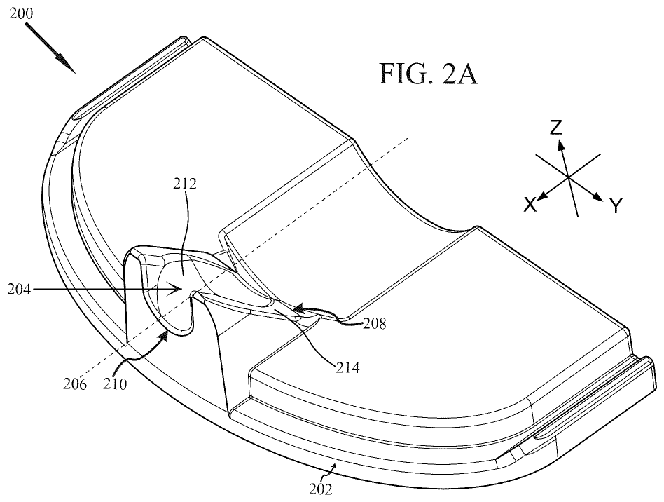

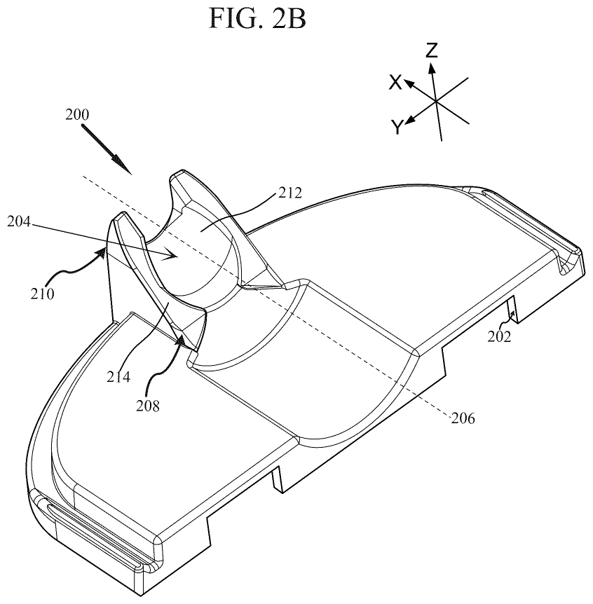

As will be further appreciated with respect to the discussion of the base 200 below, see FIGS. 2A and 2B, it will be understood and appreciated that the first section 116 of the tapered member 114, is an external and exposed surface, and is further understood and appreciated as an external engaging surface 120. It will also be appreciated that this external engaging surface 120 is not a blunt end normal to the first longitudinal axis 110.

With respect to this description, and the statement that the first portion 116 is angled up and away from the outlet to the second portion 118, it will of course be understood and appreciated that with these elements now defined, with respect to the direction of liquid flow thorough the tube head adapter 102 from the inlet 104 to the outlet 106, the tapered member 114 is indeed tapered downward towards the outlet 106.

For at least one embodiment, tube head adapter 102 also includes a disc element 122 generally normal to the first longitudinal axis 110 and disposed between the inlet 104 and the outlet 106. More specifically, the disc element 122 is disposed generally about the midsection of the tube head adapter 102 with the tapered member 114 extending from disc element 122 side opposite from the inlet 104.

As may be more fully appreciated in the plain side view of FIG. 1C and a front plain view of FIG. 1D, it will be understood and appreciated that the tapered member 114 rises circumferentially about the tube head adapter 102 from the outlet 106 and towards the inlet 104, the first portion 116 of which is angled at an acute internal angle 124 relative to the first longitudinal axis 110, and a corresponding obtuse external angle 126 relative to the first longitudinal axis 110. As the tapered member 114 is formed from generally solid material(s), and this is an external surface, with respect to the angle of external engaging surface 120, reference to the obtuse external angle 126 may be easier to appreciate.

The acute internal angle 124 and corresponding obtuse external angle 126 may be further appreciated in FIG. 1C. For at least one embodiment the obtuse external angle 126 of external engaging surface 120 is between about 91° and 179°, and more preferably between about 120° and 175°. With respect to the acute internal angle 124 of external engaging surface 120 for this same embodiment the acute internal angle 124 is between about 1° and 89°, and more preferably between about 15° and 60°.

As may be more fully appreciated in the plain front view of FIG. 1D, for at least one embodiment, the tapered member 114, and specifically the external engaging surface 120 portion of the tapered member 114, has a generally circular cross section normal to the tube head adapter 102, and more specifically the first longitudinal axis 110. As shown, the external engaging surface 120 is appreciated to be a straight linear surface.

For alternative embodiments the external engaging surface 120 may be provided by a plurality of angled surface 128 and/or curved surfaces, of which angled surfaces 128A. 128B, 128C and 128D are exemplary. Moreover, for at least one embodiment the external engaging surface 120 may be appreciated as being convex. As used herein, the term “convex” as applied to the external engaging surface 120 will be understood and appreciated to include embodiments wherein the external engaging surface 120 is provided by a plurality of different straight section surfaces which collectively provide a “rounded” profile.

It may also be appreciated most clearly in FIGS. 1D and 1E, that the disc element 122 has a larger diameter than the tapered member 114, such that with respect to the first longitudinal axis 110 the disc element 122 extends elevationally beyond the tapered member 114. In addition, for at least one embodiment the disc element 122 also has at least one flat section 130, of which flat sections 130A and 130B are exemplary. When the TLC 100 is set on a surface, the at least one flat section 130 advantageously limits the ability of TLC 100 to roll away.

The uniformity of the flared member cross section, and specifically the cross section of the external engaging surface 120 will generally be preferred as the uniformity permits the use of the TLC 100 without imposing a specific requirement for alignment when disposed upon the base 200 as described below (see FIG. 2A). Freedom from alignment is generally desirable as the luer lock fitting 112 is engaged by twisting, and a rotational alignment constraint as between the TLC 100 and the base 200 might unintentionally loose the luer lock fitting 112 engagement with a syringe.

As may also be appreciated from FIGS. 1A-1D, for at least one embodiment, the tube head adapter 102 provides at least one gripper 132 structured and arranged for gripping by an operator, such as when the operator is affixing the tube head adapter 102 by its luer lock fitting 112 to a syringe. For at least one embodiment, the at least one gripper 132 is provided as a plurality of longitudinal grooves 134 in the tapered member 114.

More specifically, for at least one embodiment about six longitudinal grooves 134 are symmetrically disposed in the outer surface of the tapered member 114, though in alternative embodiments there may be more or fewer longitudinal grooves 134 without departing from the scope of the present invention.

For at least one embodiment, the outer surface of the tapered member 114 is substantially smooth, if not slick, for ease in fabrication, for ease in engaging with the base 200 element of a pump (described further below) and to diminish the opportunity for the collection of debris. But a smooth and/or slick outer surface can be difficult to grip firmly by some operators. Accordingly, these longitudinal grooves 134 are dimensioned so as to receive portions of the operator's finger tips and/or thumb tip thus providing abutment surfaces that permit the operator to establish a firm grip upon the tube head adapter 102. And of course, for yet other embodiments, a textured surface and/or the use of other physical structures other than longitudinal grooves 134 may be provided as at least one gripper 132.

Moreover, it will be understood and appreciated that for at least one alternative embodiment, the longitudinal grooves 134 may spiral about the first longitudinal axis 110 as opposed to being parallel thereto as shown for ease of illustration and discussion. Moreover, in varying embodiment, the at least one gripper 132 may be manifested as continuous grooves, segmented grooves, radial grooves, bumps, holes, texturization, adhesive, or other elements or other such structures that permit an operator to engage his or her fingers and thumb with the tapered member 114 more substantially than may be obtained with simply a smooth outer surface.

The disc element 122 may also be used as a gripper 132 by some operators, as well as permitting the present invention to be utilized in infusion systems that expect a tube head adapter 102 with a disc element 122 for seating and/or engagement. Moreover, the disc element 122 advantageously permits embodiments of the new and improved tube head adapter 102 to be used by legacy infusion systems.

For ease of discussion and illustration, various sections/elements of the TLC 100 are shown and discussed as specific elements (tube head adapter 102, tapered member 114, disc element 122, gripper 132, etc. . . . ) but it will be understood and appreciated that these distinctions are not intended as specific limitations. Indeed, the one or more of such elements may inseparably integrated, such as an embodiment where the TLC 100 is cast or 3D printed as a unitary structure. Moreover, the use of distinct element titles is intended to facilitate an appreciation for the arrangement of the elements with respect to each other and not by way of limitation.

This tapered member 114 of the tapered lure connector provides at least four distinct advantages:

First advantage: the tapered member 114 with or without the disc element 122 provides several advantages to the TLC 100. For example, as is clear from the associated Figures, the tapered member 114 extends significantly away from the tube head adapter 102. As such, when the TLC 100 is disposed horizontally adjacent to a surface, the luer lock fitting 112 and the inlet 104 will more likely than not be held aloft, and thereby maintain a generally sterile state as they have not been subjected to surface contaminants.

Second advantage: the tapered member 114 with or without the disc element 122 provides a convenient brace for an operator to place the tips of his or her fingers and thumb when engaging or disengaging the luer lock fitting 112 to a syringe or other device.

Third advantage: the tapered member 114, and more specifically the external engaging surface 120, advantageously permits the TLC 100 to impose an elevated degree of certainty that proper tubing is used with a proper syringe pump. And that the proper tubing is appropriately retained/seated within the proper syringe pump.

Fourth advantage: in contrast to legacy systems reliant upon the disc element to seat the luer within the pump, the TLC 100 provides a greater external engaging surface 120 for a more secure and stable engagement as between the TLC 100 and the pump (not shown).

Moreover, for at least one embodiment the TLC 100 may be summarized as a tube head adapter 102 formed from a resilient material providing a luer lock fitting 112 structured and arranged to engage a syringe, and a tapered member 114 disposed at least proximately to the outlet 106, at least a portion of the tapered member 114 angled down towards the outlet 106 and providing an external engaging surface 120 about a portion of the tube head adapter 102 proximate to the outlet 106, the external engaging surface 120 obtusely angled away from the outlet 106 with respect to the first longitudinal axis 110.

For yet another embodiment, TLC 100 may be described as having a tube head adapter 102 formed of a resilient material providing: an inlet 104 and opposite thereto an outlet 106 with a first longitudinal axis 110 there between; a disc element 120 generally normal to the first longitudinal axis 110 and disposed between the inlet 104 and the outlet 106; a luer lock fitting 112 formed proximate to the inlet 106 and structured and arranged to engage a syringe; and a tapered member 114 disposed between the disc element 122 and the outlet 106, the tapered member 114 providing an external engaging surface 120 about a portion of the tube head adapter 102 proximate to the outlet 106, the external engaging surface 120 obtusely angled away from the outlet 106 with respect to the first longitudinal axis.

Moreover, with respect to the figures and this accompanying description, it will be clearly understood and appreciated that the tapered member 114 with or without the disc element 122, is disposed behind the luer luck fitting 112, such that the luer lock fitting 112 is free to engage a corresponding luer lock fitting provided by a syringe or other device, unencumbered. Perhaps of even greater importance, whether attached to a syringe or other device or not, the luer lock fitting 112 does not impede or otherwise directly affect the tapered member 114 from being mated to a base 200 with a corresponding receiving surface structured and arranged to receive the external engaging surface 120 of the TLC 100.

It will also be understood and appreciated that in varying embodiments, the diameter of the luer lock fitting 112 maybe larger or smaller than the diameter of the tube head adapter 102 extending beyond the tapered member 114 and/or the disc element 122 to the outlet 106. Indeed, the use of different diameters for the luer lock fitting 112 and/or the tube head adapter 102 extending beyond the tapered member 114 and/or the disc element 122 to the outlet 106 may advantageously permit different TLC 100s specifically configured for distinct uses.

As noted above, in FIGS. 1A-1E the TLC 100 has been shown free of medical tubing for ease of illustration and discussion. In practice, the outlet 106 of the TLC 100 is bonded, joined or otherwise fused to medical tubing such that engaging the luer lock to a syringe or other device will permit that syringe or other device to introduce a liquid or gas through the TLC 100 and into the medical tubing.

In FIGS. 2A and 2B there is shown a base 200 in accordance with at least one embodiment of the present invention. More specifically, the base 200 is provided by a body of resilient material 202 with a longitudinal groove 204 therein structured and arranged to receive at least a portion of the TLC 100, and more specifically at least a portion of the tapered member 114.

Indeed, as shown in FIGS. 2A and 2B, it will be understood and appreciated that the base 200 provides a longitudinal groove 204 along a second longitudinal axis 206, the longitudinal groove 204 having a first end 208 and opposite thereto a second end 210. It will also be appreciated that the longitudinal groove 204 tapers from the first end 208 towards the second end 210.

Disposed between the first end 208 and the second end 210 of the longitudinal groove 204 is an inner receiving surface 212 tapering towards the second longitudinal axis 206 and the second end 210. The inner receiving surface 212 is angled to receive at least a portion of the external engaging surface 120 as provided by a TLC 100 described herein. More specifically, the inner receiving surface 212 is angled at a corresponding angle to receive the external engaging surface 120 of a TLC 100.

Moreover, for a TLC 100 with an external engaging surface 120 angled at an obtuse external angle 126 being between 120° and 175°, the inner receiving surface 212 is angled to a corresponding internal angle 124 of between 60° and 15°. For at least one embodiment the obtuse external angle 126 of the external engaging surface 120 is about 170° and the corresponding acute interior angle of the inner receiving section is about 20° relative to second longitudinal axis 206.

As with the external engaging surface 120 as described above, for at least one embodiment the inner receiving surface 212 may be appreciated as being concave, as it is tapering inward and from the first end 208 towards the second end 210. Further still, though shown to be a straight linear surface, for at least one embodiment the inner receiving surface 212 may be provided by a plurality of angled surfaces and/or curved surfaces. As used herein, the term “concave” as applied to the inner receiving surface 212 will be understood and appreciated to include embodiments wherein the inner receiving surface 212 is provided by a plurality of different section surfaces which collectively provide a “rounded in” or “depressed” profile.

For ease of illustration and discussion, the exemplary base 200 shown in FIGS. 2A and 2B is shown as a component of a larger structure having a platform. It will be understood and appreciated that the base 200 may be incorporated as a component of a syringe pump. For at least one embodiment, the base 200 may be provided as an insert with at least one attacher (such as, but not limited to a snap fit connector) such that it may be disposed into an existing syringe pump so as to permit operation of the syringe pump in connection with the TLC 100, with a base 200 that was originally provided for receiving legacy lure connectors/legacy tube head adapters. Of course, for at least one embodiment, the base 200 is provided as an integral component of the syringe pump system.

Disposed about the receiver surface of the base 200 is a deflector section 214. More specifically, as may be appreciated from at least FIGS. 2A and 2B, the deflector section 214 slops across the longitudinal groove 204 between the first end 208 and the second end 210. Collectively, the inner receiving surface 212 and the deflector section 214 are structured and arranged to ensure that the base 200 can and will properly receive a TLC 100 that provides a corresponding external engaging surface 120 and reject other luer connectors.

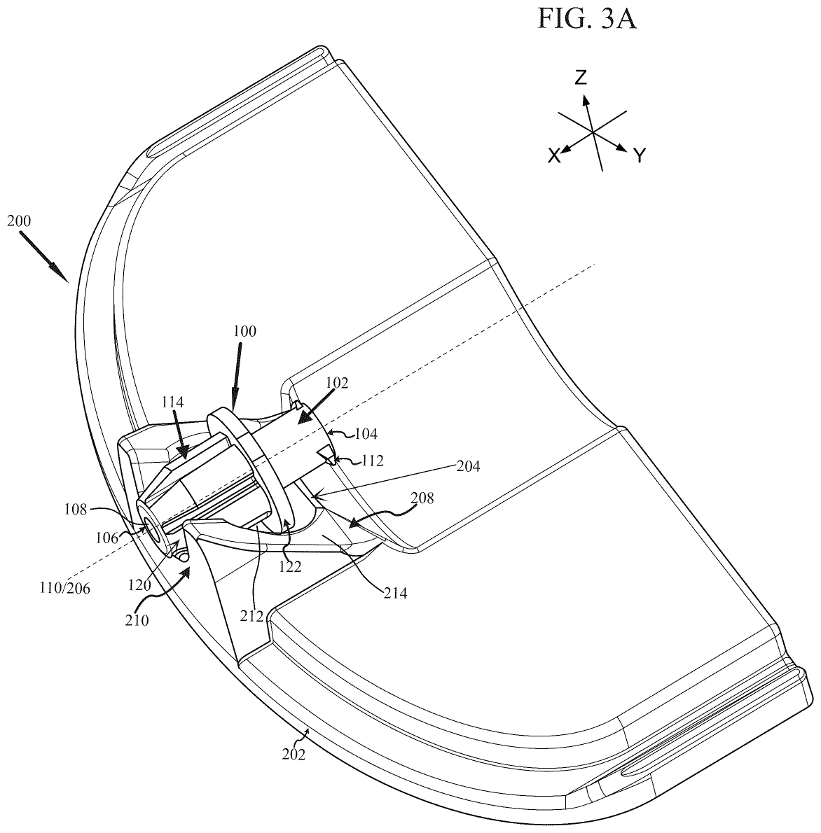

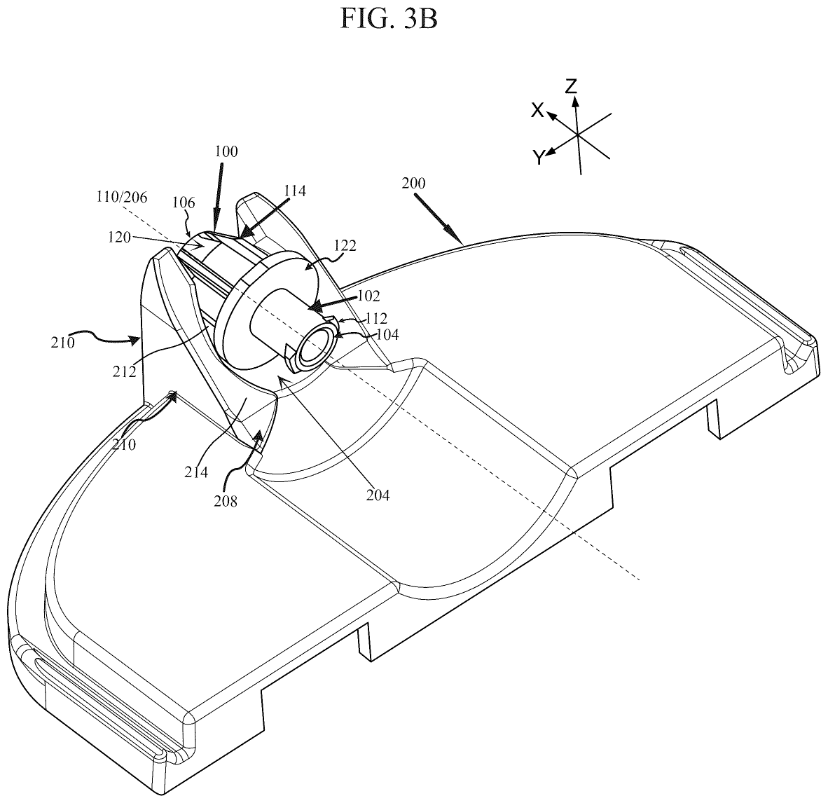

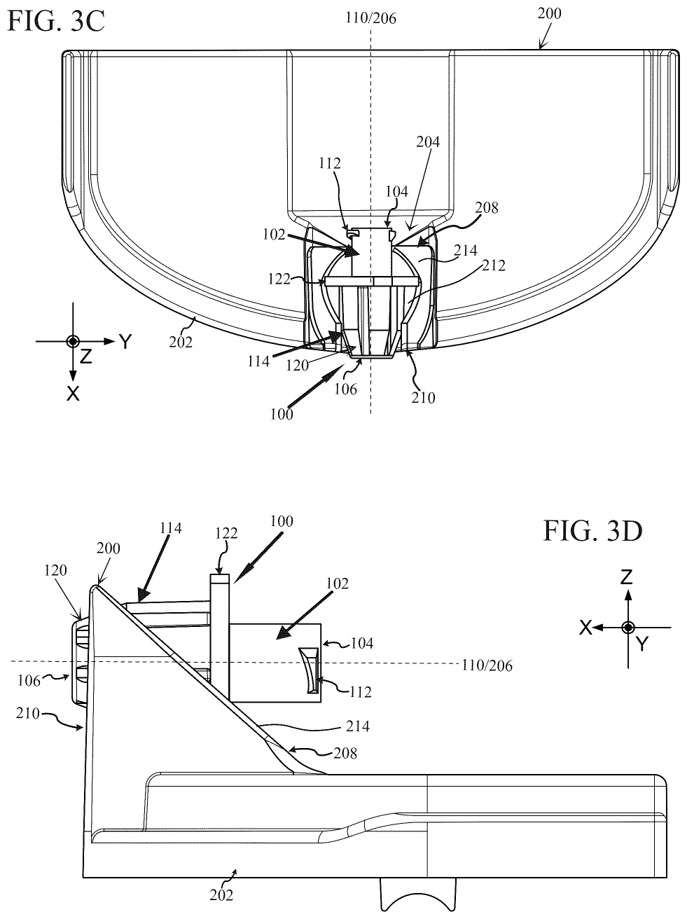

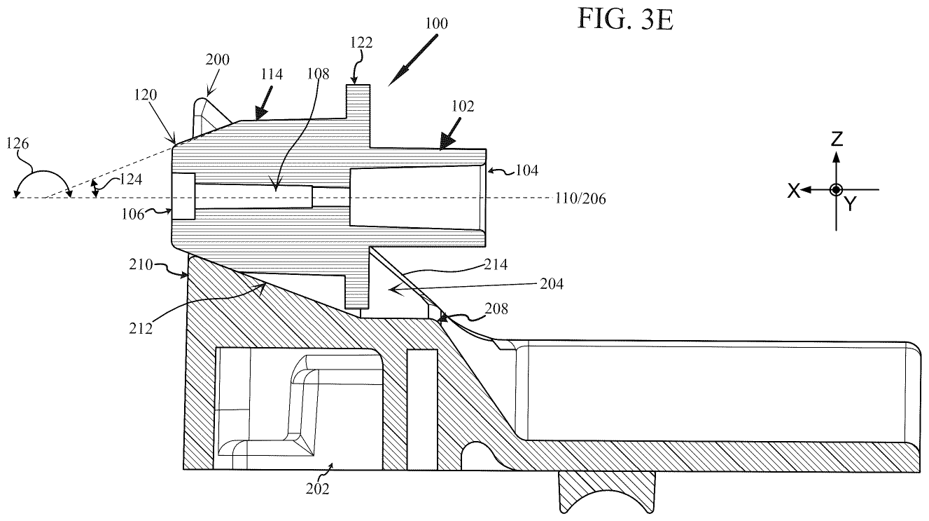

FIG. 3A provides an upper front perspective view of a TLC 100 received and seated within the base 200, and FIG. 3B provides an upper rear perspective view of a TLC 100 received and seated within the base 200. FIG. 3C provides a top plain view of a TLC 100 received and seated within the base 200, and FIG. 3D provides a plain side view of a TLC 100 received and seated within the base 200. In each of FIGS. 3A-3D, a syringe coupled to the TLC 100 has not been shown so as to more fully appreciate the relationship between the TLC 100 and the base 200.

FIG. 3E provides a side cut through plain view of a TLC 100 received and seated within the base 200, as shown in FIG. 3D, further permitting appreciation of the engagement between the external engaging surface 120 of the TLC 100 and the inner receiving surface 212 of the base 200.

With respect to FIG. 3E, it may also be appreciated that for at least one embodiment the disc element 122 is not in physical contact with the inner receiving surface 212. More specifically, the snug engagement of the TLC 100 to the base 200 is achieved entirely between the external engaging surface 120 of the TLC 100 and the inner receiving surface 212 of the base 200.

With respect to FIG. 3E it may be understood and appreciated, that for at least one embodiment of the present invention, a greater area of contact between the external engaging surface 120 and the inner receiving surface 212 is achieved in the lower portion of the base 200 than in the upper portion of the base 200.

As may be understood and appreciated with respect to FIGS. 3A and 3B, the longitudinal groove 204 is fully open directly above longitudinal axis 206. For a horizontal cross section including the longitudinal axis 206 in the X-Y plane, the side portions of the inner receiving surface 212 of the base 200 that engage with the external engaging surface 120 of the TLC 100 may be substantially symmetrical. However, for angled cross sections including the longitudinal axis 206, a first portion of the inner receiving surface 212 has a first length and opposite thereto a second portion of the inner receiving surface 212 has a second length, the first length longer than the second length. While this difference in points of contact, i.e., length of contact surface, is not an issue when the base 200 receives an intended TLC 100, it does advantageously assist the base 200 with rejecting an unintended luer connector.

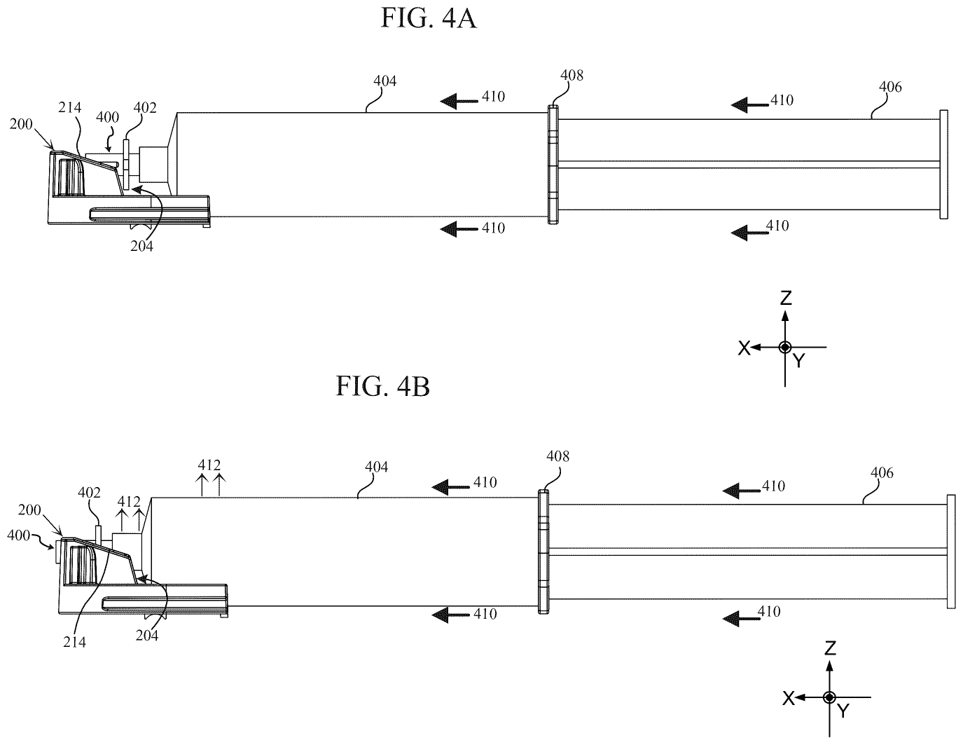

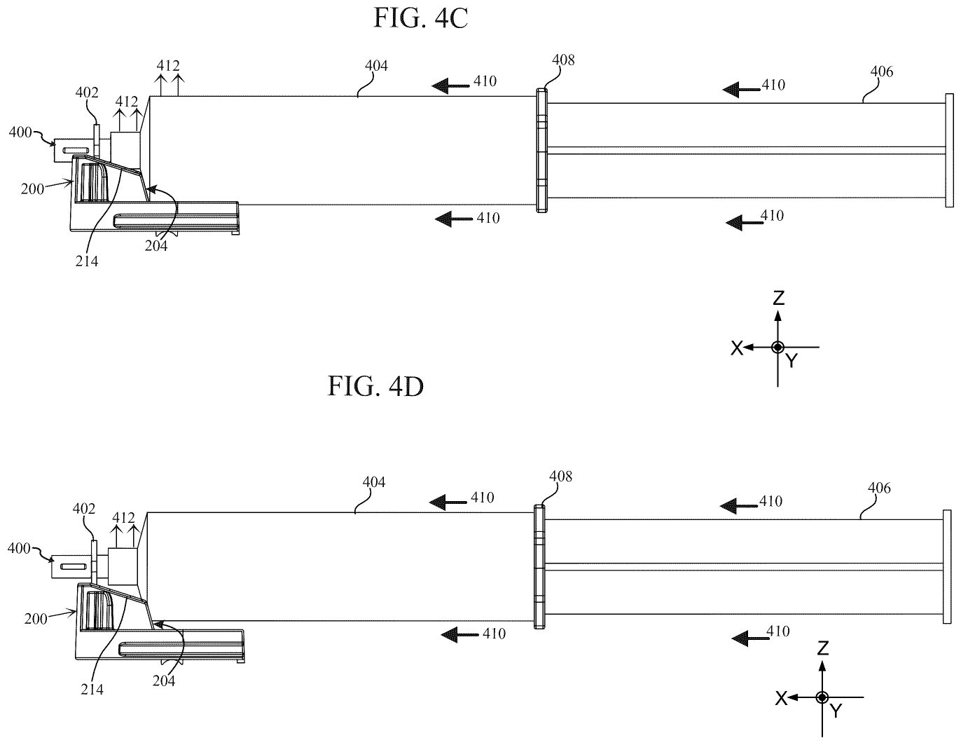

Moreover, FIGS. 4A-4D provide progressive side views of an attempt to dispose a legacy luer connector 400 with a legacy disc 402 attached to a syringe 404 within base 200, with the base 200 advantageously deflecting the legacy luer 400 up and out of the base 200. More specifically, as shown initially in FIG. 4A, as force is applied to the syringe 404 via the plunger 406 in the barrel or finger tabs 408, the syringe is driven forward as indicated by arrows 410.

As shown in FIG. 4B, this forward motion advances the syringe 404 and more specifically the legacy luer connector 400 towards the longitudinal groove 204. The legacy disc 402 is not compliant, and connects with the outer edge deflector section 214, as a result the legacy luer connector 400 is driven up, as shown by arrows 412.

As shown in FIGS. 4C and 4D, as the syringe 404 continues to move forward, shown by arrows 400, the deflector section 214 continues to direct the legacy lure connector 400 up, and eventually out over the top of the base 200, thereby advantageously preventing engagement of the non-compliant legacy lure connector 400 with the base 200.

Although this description is set forth with force being applied to the plunger, it will also be understood and appreciated that the same result is achieved if force is applied to drive the base 200 towards the nozzle of the syringe 402 to which the legacy luer connector 400 is affixed. More specifically relative motion as between the syringe 402 and the base 200 will result in a legacy luer connector 400 being ejected whereas an intended embodiment of the TLC 100 will be received and properly seated.

Those skilled in the art will further understand and appreciate that the advantageous relationship between a corresponding external engaging surface 120 and an inner receiving surface 212 may further permit advantageous embodiments where a TLC 100 and corresponding base 200 further ensure that intended medications may be dispensed from a syringe pump while unintended medications may not. More specifically, the use of proper corresponding angles as between the external engaging surface 120 and the inner receiving surface 212 can ensure that only syringes with properly correlating TLC 100s may be seated in certain base 200s, with any non-correlating luers-tapered or legacy-being ejected.

The base 200 and tubing having a TLC 100 may be provided as a kit, the kit prepared and provided in a sterile packaging for use when and as desired.

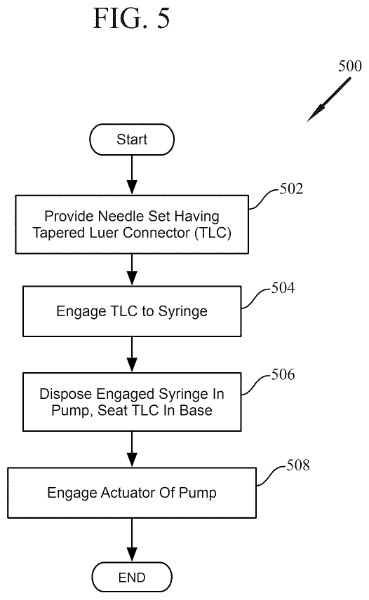

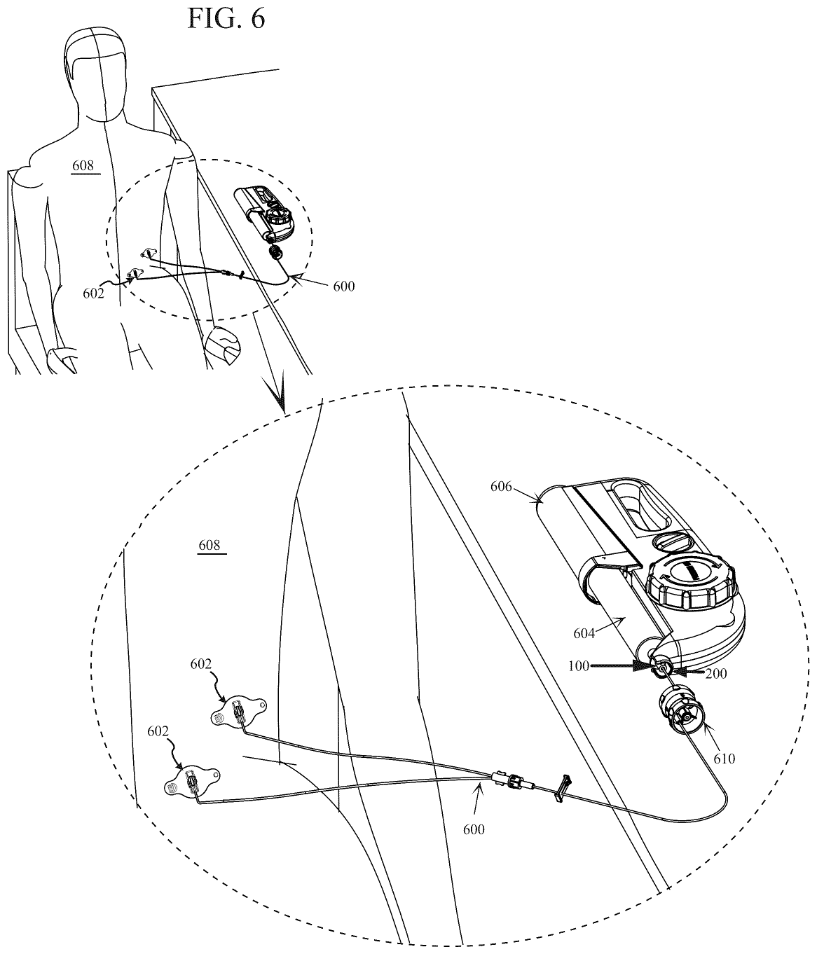

Having described embodiments of the TLC 100, other embodiments relating to at least one method 500 of using a TLC 100 will now be discussed, see FIG. 5, in connection with an infusion system 600 shown in FIG. 6. It will be appreciated that the described method need not be performed in the order in which it is herein described, but that this is merely exemplary of one method of using a TLC 100.

In general, the method 500 commences with providing a needle set 600 having at least a TLC 100 and one or more needles 602. As noted above, the TLC 100 has a tube head adapter 102 formed of a resilient material 202 having an inlet 104 and opposite thereto an outlet 106 with a first longitudinal axis 110 therebetween, block 502. The tube head adapter 102 further provides a luer lock fitting 112 proximate to the inlet 104 and structured and arranged to engage a syringe.

As described above, the tube head adapter 102 has a tapered member 114 disposed at least proximately to the outlet, at least a portion of the tapered member 114 angled down towards the outlet and providing an external engaging surface 120 about a portion of the tube head adapter 102 proximate to the outlet 106, the outer engaging surface obtusely angled away from the outlet 106 with respect to the first longitudinal axis 110, the engaging surface structured and arranged to engage a correspondingly angled receiver surface of a base 200.

The needle set 600 provides needles 602, such as butterfly needles set forth in U.S. patent application Ser. No. 18/216,342 entitled SYSTEM AND METHOD FOR BUTTERFLY NEEDLE ASSEMBLY, incorporated herein by reference. Further still, these needles 602 may be vented needles as set forth in U.S. patent application Ser. No. 18/760,544, entitled SYSTEM AND METHOD FOR VENTED NEEDLE ASSEMBLY, incorporated herein by reference.

The method 500 continues with engaging the needle set 600 to a syringe 604 by way of the TLC 100. More specifically, the luer lock fitting 112 of the TLC 100 is engaged to a syringe 604 having a barrel with a plunger disposed within and movable along the barrel, the tapered member 114 thus extending away from the syringe, block 504.

Next, the syringe 604 with engaged needle set 600 is disposed within a pump 606 having an actuator and a base 200, block 506. As described above, the base 200 has a longitudinal groove 204 therein structured and arranged to receive at least a portion of the TLC 100.

For at least one embodiment, the pump 606 is a constant pressure pump 606, such as the Freedom60® Syringe Infusion Pump or the FreedomEdge Syringe Infusion Pump as provided by KORU Medical Systems, Inc. of Mawah, New Jersey, provides base 200 as described above.

As shown in FIG. 6, the needles 602 have been disposed into a patient 608 in the lower torso area. The needle set 600 may or may not include an optional flow controller 610.

The method 500 continued with engaging the actuator of the pump 606, which when activated is structured and arranged to engage the plunger of the syringe 604 and drive the plunger towards the tubing, block 508. The force of the actuator further engaging the external engaging surface 120 of the TLC 100 with the inner receiving surface 212 of the base 200, to securely seat the syringe 604 to the base 200 of the pump 606.

Changes may be made in the above methods, systems and structures without departing from the scope hereof. It should thus be noted that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A tapered luer connector for medical tubing comprising:
a tube head adapter formed of a resilient material providing:
an inlet and opposite thereto an outlet with a longitudinal axis there between;
a luer lock fitting formed proximate to the inlet and structured and arranged to engage a syringe; and
a tapered member disposed at least proximately to the outlet, at least a portion of the tapered member angled down towards the outlet and providing an external engaging surface about a portion of the tube head adapter proximate to the outlet, the external engaging surface obtusely angled away from the outlet with respect to the longitudinal axis.

2. The tapered luer connector of claim 1, wherein the tube head adapter further provides at least one gripper structured and arranged for gripping by an operator as the luer lock fitting is engaged with a syringe.

3. The tapered luer connector of claim 2, wherein the gripper is a disc element generally normal to the longitudinal axis extending radially about the tapered member opposite from the outlet.

4. The tapered luer connector of claim 2, wherein the gripper is a plurality of longitudinal grooves in the tapered member extending from the outlet towards the inlet.

5. The tapered luer connector of claim 2, wherein the gripper is at least one element selected from the group consisting of: continuous grooves, segmented grooves, radial grooves, bumps, holes, texture, adhesive.

6. The tapered luer connector of claim 1, further including a disc element generally normal to the longitudinal axis and joined to the tapered member opposite from the outlet.

7. The tapered luer connector of claim 1, wherein the angle of the external engaging surface is constant.

8. The tapered luer connector of claim 1, wherein the external angle of the external engaging surface relative to the longitudinal axis is selected to be between about 91° and 179°, and more preferably between about 120° and 175°.

9. The tapered luer connector of claim 1, wherein the internal angle of the external engaging surface relative to the longitudinal axis is selected to be between about 1° and 89°, and more preferably between about 15° and 60°.

10. The tapered luer connector of claim 1, wherein the external engaging surface is convex.

11. A tapered luer connector for medical tubing comprising:
a tube head adapter formed of a resilient material providing:
an inlet and opposite thereto an outlet with a longitudinal axis there between;
a disc element generally normal to the longitudinal axis and disposed between the inlet and the outlet;
a luer lock fitting formed proximate to the inlet and structured and arranged to engage a syringe; and
a tapered member disposed between the disc element and the outlet, the tapered member providing an external engaging surface about a portion of the tube head adapter proximate to the outlet, the external engaging surface obtusely angled away from the outlet with respect to the longitudinal axis.

12. The tapered luer connector of claim 11, wherein the tube head adapter further provides at least one gripper structured and arranged for gripping by an operator as the luer lock fitting is engaged with a syringe.

13. The tapered luer connector of claim 12, wherein the gripper is a plurality of longitudinal grooves in the tapered member extending from the outlet towards the disc element.

14. The tapered luer connector of claim 12, wherein the gripper is at least one element selected from the group consisting of: continuous grooves, segmented grooves, radial grooves, bumps, holes, texture, adhesive.

15. The tapered luer connector of claim 11, wherein the angle of the external engaging surface is constant.

16. The tapered luer connector of claim 11, wherein the external angle of the external engaging surface relative to the longitudinal axis is selected to be between about 91° and 179°, and more preferably between about 120° and 175°.

17. The tapered luer connector of claim 11, wherein the internal angle of the external engaging surface relative to the longitudinal axis is selected to be between about 1° and 89°, and more preferably between about 15° and 60°.

18. The tapered luer connector of claim 11, wherein the external engaging surface is convex.

19. A tapered luer connector for medical tubing comprising:
a tube head adapter formed of a resilient material providing an inlet and opposite thereto an outlet with a longitudinal axis there between and at least two members, a first member as a luer lock fitting adjacent to the inlet and structured and arranged to engage a syringe; and
a second member as a circumferential tapered member adjacent to the outlet, the circumferential tapered member having an external engaging surface proximate to the outlet and sloping away from the luer lock fitting, at least a portion of the external engaging surface structured and arranged to engage a correspondingly angled receiver section of a base, the second member having a circumferential disc element extending normally with respect to the longitudinal axis.

20. The tapered luer connector of claim 19, wherein the tube head adapter further provides at least one gripper structured and arranged for gripping by an operator as the luer lock fitting is engaged with a syringe.

21. The tapered luer connector of claim 20, wherein the gripper is a disc element generally normal to the longitudinal axis extending radially about the tapered member opposite from the outlet.

22. The tapered luer connector of claim 20, wherein the gripper is a plurality of longitudinal grooves in the tapered member extending from the outlet towards the inlet.

23. The tapered luer connector of claim 20, wherein the gripper is at least one element selected from the group consisting of: continuous grooves, segmented grooves, radial grooves, bumps, holes, texture, adhesive.

24. The tapered luer connector of claim 19, further including a disc element generally normal to the longitudinal axis and joined to the tapered member opposite from the outlet.

25. The tapered luer connector of claim 19, wherein the angle of the external engaging surface is constant.

26. The tapered luer connector of claim 19, wherein the external angle of the external engaging surface relative to the longitudinal axis is selected to be between about 91° and 179°, and more preferably between about 120° and 175°.

27. The tapered luer connector of claim 19, wherein the internal angle of the external engaging surface relative to the longitudinal axis is selected to be between about 1° and 89°, and more preferably between about 15° and 60°.

28. The tapered luer connector of claim 19, wherein the external engaging surface is convex.

29. A base comprising:
a body of resilient material with a longitudinal groove therein structured and arranged to receive at least a portion of the tapered luer connector, the longitudinal groove having:
a longitudinal centerline;
a first end and opposite thereto a second end;
an inner receiver section tapering towards the longitudinal centerline and the second end, the inner receiver section angled to receive at least a portion of an external engaging surface provided by a tapered luer connector; and
an outer edge deflector section sloping across the longitudinal groove between the first end and the second end.

30. The base of claim 29, wherein the inner receiver section is generally concave.

31. The base of claim 29, wherein a cross section of the inner receiver section normal to the longitudinal centerline is asymmetrical.

32. The base of claim 29, wherein the inner receiver section angle is selected to be between about 1° and 89°, and more preferably between about 15° and 60°.

33. The base of claim 29, wherein the inner receiver section is structured and arranged such that a luer connector without a corresponding engaging surface will be ejected.

34. A syringe pump for use with a tapered luer connector for medical tubing comprising:
a housing structured and arranged to receive a syringe having a plunger movable along a barrel and an engaged tubing, the engaged tubing providing a head adapter providing an inlet and opposite thereto an outlet with a longitudinal axis there between with a luer lock fitting proximate to the inlet engaged to the syringe and a circumferential tapered member adjacent to the outlet, the circumferential tapered member having an external engaging surface proximate to the outlet and sloping away from the luer lock fitting, the engaging surface structured and arranged to engage a correspondingly angled receiver section of a base;
a base having:
a body of resilient material with a longitudinal groove therein structured and arranged to receive at least a portion of the tapered luer connector, the longitudinal groove having:
a longitudinal centerline;
a first end and opposite thereto a second end;
an inner receiver section tapering towards the longitudinal centerline and the second end, the inner receiver section angled to receive at least a portion of an external engaging surface provided by a tapered luer connector;
an outer edge deflector section sloping across the longitudinal groove between the first end and the second end; and
an actuator structured and arranged to engage the plunger of the syringe and drive the plunger towards the tubing, the force of the actuator further engaging the external engaging surface of the tapered member to the inner receiver section of the base.

35. The syringe pump of claim 34, wherein the inner receiver section is generally concave.

36. The syringe pump of claim 34, wherein a cross section of the inner receiver section normal to the longitudinal centerline is asymmetrical.

37. The syringe pump of claim 34, wherein the inner receiver section angle is selected to be between 15° and 60°.

38. The syringe pump of claim 34, wherein the external obtuse angle of the external engaging surface relative to the longitudinal axis is selected to be between about 91° and 179°, and more preferably between about 120° and 175°.

39. The syringe pump of claim 34, wherein the internal angle of the external engaging surface relative to the longitudinal axis is selected to be between about 1° and 89°, and more preferably between about 15° and 60°.

40. The syringe pump of claim 34, wherein the inner receiver section is structured and arranged such that a luer connector without a corresponding engaging surface will be ejected.

41. A method for using tapered luer medical tubing, comprising:
providing a tube head adapter formed of a resilient material providing:
an inlet and opposite thereto an outlet with a longitudinal axis there between;
a luer lock fitting formed proximate to the inlet and structured and arranged to engage a syringe; and
a tapered member disposed at least proximately to the outlet, at least a portion of the tapered member angled down towards the outlet and providing an external engaging surface about a portion of the tube head adapter proximate to the outlet, the external engaging surface obtusely angled away from the outlet with respect to the longitudinal axis, the engaging surface structured and arranged to engage a correspondingly angled receiver section of a base;
engaging the luer lock fitting of the tubing to a syringe having a barrel with a plunger disposed within and movable along the barrel, the angled engaging surface disposed away from the syringe; and
disposing the syringe with engaged tubing within a pump having an actuator and a base, the base having:
a body of resilient material with a longitudinal groove therein structured and arranged to receive at least a portion of the tapered luer connector, the longitudinal groove having:
a longitudinal centerline;
a first end and opposite thereto a second end;
an inner receiver section tapering towards the longitudinal centerline and the second end, the inner receiver section angled to receive at least a portion of an external engaging surface provided by a tapered luer connector;
an outer edge deflector section sloping across the longitudinal groove between the first end and the second end; and
an actuator structured and arranged to engage the plunger of the syringe and drive the plunger towards the tubing, the force of the actuator further engaging the external engaging surface of the tapered member to the inner receiver section of the base.

42. The method of claim 41, wherein the tube head adapter further provides at least one gripper structured and arranged for gripping by an operator as the luer lock fitting is engaged with a syringe.

43. The method of claim 41, wherein the inner receiver section is generally concave.

44. The method of claim 41, wherein a cross section of the inner receiver section normal to the longitudinal centerline is asymmetrical.

45. The method of claim 41, wherein the inner receiver section angle is selected to be between 15° and 60°.

46. The method of claim 41, wherein the external angle of the external engaging surface relative to the longitudinal axis is selected to be between about 91° and 179°, and more preferably between about 120° and 175°.

47. The method of claim 41, wherein the internal angle of the external engaging surface relative to the longitudinal axis is selected to be between about 1° and 89°, and more preferably between about 15° and 60°.

48. The method of claim 41, wherein the inner receiver section is structured and arranged such that a luer connector without a corresponding engaging surface will be ejected.

49. The method of claim 41, wherein the base is a component of a syringe pump.

50. The method of claim 41, wherein the base is an insert structured and arranged to couple with the base housing.
